(19) United States
(12) Patent Application Publication
(10) Pub. No.: US 2002/0095210 A1
(43) Pub. Date: Jul. 18, 2002

(54) Heart pump graft connector and system

(76) Inventors: Michael T. Finnegan, North Reading, MA (US); Long S. Yu, Rocklin, CA (US)

Correspondence Address: NUTTER MCCLENNEN & FISH LLP, ONE INTERNATIONAL PLACE, BOSTON, MA, 02110, US

(21) Appl. No.: 09/761,367

(22) Filed: Jan. 16, 2001

(51) Int. Cl. A61M 1/12

(52) U.S. Cl. 623/3.26; 623/1.36; 600/16

(57) ABSTRACT

A connector for connecting a blood processing device to vascular tissue includes a vascular tissue connecting element that is suturable to a portion of the cardiovascular system for providing a flow connection. A junction ring is affixed to the vascular tissue connecting portion in order to form a substantially shape retaining connecting element. A locking ring for locking the junction ring to a blood processing device includes a coupling element configured to engage a port on the blood processing device so that rotation of the locking ring draws the junction ring to the port and locks the junction ring into a sealing relationship with the port. The locking ring is freely rotatable about the junction ring so that the locking ring can be rotated to lock the junction ring to the port without twisting the vascular tissue connecting element.

### BACKGROUND OF THE INVENTION

The present invention relates to medical flow connectors, and in particular to flow connectors for an implantable, pressurized flow device such a ventricular assist device or a blood pump. The development of a suitable design for such a pump has occupied researchers at many institutions for the better part of several decades, requiring meticulous engineering research and breakthroughs in biocompatible materials, pump/motor construction, and graft development, as well as research and development on suitable junctions and valves for connecting the devices to the vasculature or to vestigial heart tissue.

By way of example, atrial cuffs and vascular graft connector junctions, as well as mountings for artificial valves which may be included in such structures, have been identified as primary loci for thrombus formation. This is due to many factors, including the surrounding fluid flow conditions, physical gaps and irregularities in the structures, and also the surface biocompatibility properties of the materials employed in such structures.

The foregoing problems of graft/connector construction have been addressed by several approaches, including the precision formation of mating structural elements to eliminate gaps; the micro-finishing of exposed surfaces; design of suitable flow passage and chamber shapes to discourage microembolus formation; and the selection of appropriately biocompatible materials. One suitable construction addressing these issues is shown in U.S. Pat. No. 5,084,064 issued Jan. 28, 1992 to Jacob Barak et al. That patent and the documents referred to therein are hereby incorporated by reference for their specific descriptions of desirable material coating, surface properties, and flow passage constructions.

In general, it may be said that the connection of such a cuff or graft to an artificial heart involves the connection of a flexible fabric or polymer sheet or tube to a rigid mechanical assembly, and this has been typically effected by building a suitable rigid termination, such as a threaded collar, onto the end of the flexible cuff or graft component, and attaching it to a port of the heart or assist pump assembly. Thus, for example, the aforesaid '064 patent shows a cone-shaped fabric cuff bonded to a rigid tube/collar ending. When surgically implanted, this rigid termination is generally threaded onto the pump device to assure permanent integrity of the junction once the device has been implanted. Other forms of attachment, such as a tooth and groove or detent, a circle clip or a clamp ring have been proposed for implementing the junction between the pump and the flow conduit.

It will be understood that surgical installation of a blood pump using such graft connectors requires the surgeon to accurately lay out the cuff, trim it as appropriate, and suture it to the remnant of atrial tissue; he or she must also carry out similar trimming, aligning and suturing of the vessel graft connector to the aorta, such that the two sewn-on connectors lie in positions to connect to the rigidly-spaced pump ports. The rigid inlet and outlet ports of the artificial heart device are then connected to the two connectors and the device is tucked into the chest cavity to sit in a natural, i.e. a non-protruding and protected but unstressed, position in the thoracic cavity. As described in the aforesaid '064 patent, a temporary holder or jig may be used to assist in aligning the grafts as they are sutured to vascular tissue prior to their connection to the pump. However, it has been found that, once these grafts are sutured to tissue, difficulty is still experienced with respect to the rotational alignment of each graft about the axis of its generally cylindrical rigid connector, and this may lead to pulling, twisting or other stressed displacement of the graft and/or of the tissue to which it is sutured.

### SUMMARY OF THE INVENTION

The present invention provides a connector for connecting a medical device, such as a heart pump or the like, to the cardiovascular system of a patient. In one aspect of the invention, a connector for connecting a blood processing device to vascular tissue is provided. The connector includes a vascular tissue connecting element that is suturable to a portion of the cardiovascular system for providing a flow connection. A junction ring is affixed to the vascular tissue connecting portion in order to form a substantially shape retaining connecting element. A locking ring for locking the junction ring to a blood processing device includes a coupling element configured to engage a port on the blood processing device so that rotation of the locking ring draws the junction ring to the port and locks the junction ring into a sealing relationship with the port. The locking ring is freely rotatable about the junction ring so that the locking ring can be rotated to lock the junction ring to the port without twisting the vascular tissue connecting element. In this way, the connector element can be sutured to the patient's tissue, then locked into a sealing relationship with the blood processing device without applying a rotational stress on the connector-tissue interface.

In another aspect of the invention, a medical quick connector for making a fluid connection between vascular tissue and a medical device is provided having a first connector half and a second connector half. The first connector half has a mating end and a connector engaging end including a junction ring affixed thereto. A locking ring is rotatably coupled to the junction ring. The second connector half has a mating end and a connector engaging end having a locking ring coupling element. A first one of the mating ends of the first and second connector halves is adapted to mate with vascular tissue, while a second one of the mating ends of the first and second connector halves is adapted to mate with the medical device. In this quick connector, the first connector half can be aligned with the second connector half, placed into engagement with the second connector half, then locked to the second connector half by rotation of the locking ring without rotation of either the first or second connector halves.

In a further aspect of the invention, a connector for connecting a blood pumping device to vascular tissue is provided. The connector includes a vascular tissue connecting element that is suturable to a portion of a patient's cardiovascular system for providing a flow connection and a junction ring defining a central opening affixed to the vascular tissue connecting portion to form a substantially shape retaining connecting element. A cross member is provided extending across a central opening of the junction ring, the cross member being configured to prevent vascular tissue from collapsing into the central opening. A locking ring is also provided for locking the junction ring to a blood pumping device.

In a still further aspect of the invention, a medical device and connector system for attaching the medical device having a port formed thereon to a cardiovascular system is provided. This system includes a connector element having a vascular tissue connecting portion and a junction ring attached to the vascular tissue connecting portion and having a predefined shape defining a central opening. A locking ring is provided on a first one of the port and the connector element and a locking ring engaging element provided on a second one of the port and the connector element. The locking ring is freely rotatable about the junction ring so that the locking ring can be rotated to lock the junction ring to the port without twisting the connecting element.

In specific embodiments, the locking ring employed in the various aspects of the invention can include a plurality of protrusions directed radially inward for engaging a locking ring engaging element to draw the junction ring into sealed connection upon rotation of the locking ring. The locking ring can have a first surface positioned to axially bear against the junction ring and exert force thereon, yet still be freely rotatable about the junction ring so as to bring the protrusions into engagement with the locking ring engaging element to draw the first surface in a direction along the flow axis of the connector. Once the ring is fully rotated, a surface feature or edge of the protrusions is configured to engage the device to prevent further rotation of the locking ring, while maintaining the axial force on the coupling. This forms a secure and fluid-tight connection to the device without prematurely fixing the orientation of, or introducing twist in, the vascular tissue connection.

In different embodiments, the vascular tissue connecting element is a vascular graft or an atrial cuff, and these can connect to outflow and inflow ports, respectively, of a blood pumping device. Preferably, the protrusions of the locking ring are symmetrically spaced at pairwise opposed positions about its circumference to engage the device and exert a uniform force on the junction ring. A seal such as an O-ring may be placed in a series pressure relationship with the junction ring and a first surface of the junction ring to compressibly distribute the force exerted on the junction ring, thus assuring that a sufficient force is applied without creating excessive localized stress in the compressed coupling. The seal ring may also be positioned to function as a fluid seal for the junction as the axial force is applied, for example, by locating it between the junction ring and the device.

### BRIEF DESCRIPTION OF THE DRAWINGS

These and other features of the invention will be understood from the description herein and illustrative figures showing representative embodiments of the invention, together with the background art such as is known to those of ordinary skill in the field, wherein

FIG. 1 is a perspective view of an artificial heart blood pump with an atrial cuff inlet connector and a vascular graft outlet connector of the present invention;

FIG. 2 is perspective view from inside a patient's atrium of the atrial cuff connector shown in FIG. 1;

FIG. 3 is a side view of the blood pump inlet port and atrial cuff connector of FIG. 1;

FIGS. 4A-4C are successive cross-section views of the blood pump inlet port and atrial cuff connector of FIG. 3 in various stages of coupling;

FIG. 5 is an end view (from the bottom or locking tab side) of a locking ring useful with the atrial cuff connector of FIG. 3; and

FIG. 6 is a side view with partial cross-section of the blood pump outlet port and vascular graft connector of FIG. 1.

### DETAILED DESCRIPTION

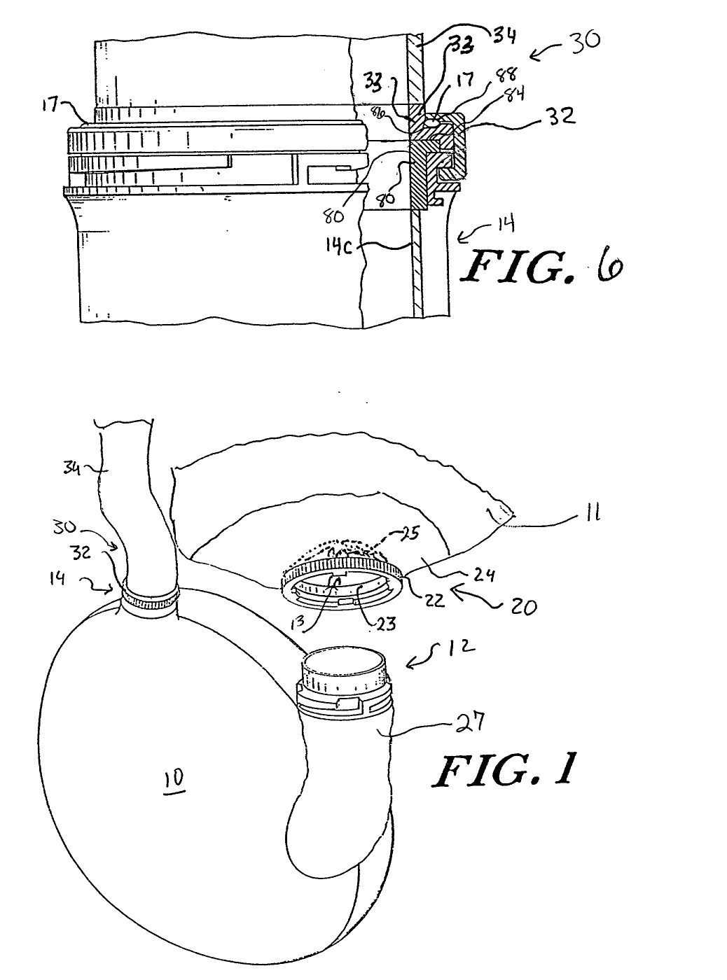

The present invention is a novel connector for attaching vascular tissue to a blood processing device such as an artificial heart. FIG. 1 illustrates one embodiment of the invention, wherein an inlet vascular tissue connector 20 is configured to connect a remaining portion of a patient's atrium 11 to the inlet port 12 of a heart pump 10. As shown in that FIG., the heart pump 10 is a relatively small assembly sized to fit within the thoracic cavity, and is illustratively embodied in a small or disk-like shape with an internal impeller (not shown) that drives blood from the inlet port 12 to an outlet port 14 located about ten centimeters from the inlet. The invention relates to a secure structure and method for attaching vascular tissue connectors to such pump ports, or to valve structures attached to the ports, which allows substantially stress-free alignment of the pump and connector, yet assures that the junction does not loosen or become undone once the pump is implanted. The term vascular tissue connector is understood to include a prosthetic structure which can be attached to the patient's natural tissue, such as a portion of a heart or an artery, and which also has a substantially rigid or shaped termination component connectable to a substantially rigid device port, e.g., the rigid heart pump assembly. A vascular tissue connector is thus a form of hybrid person-to-machine coupling interface.

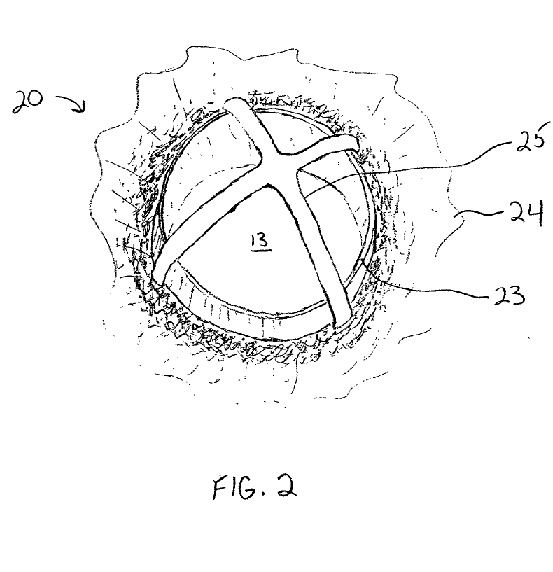

For exemplary heart pump 10 of FIG. 1, the vascular tissue inlet connector 20 for coupling to inlet port 12 is an atrial cuff assembly. Atrial cuff assembly 20 is formed of a conical skirt or cuff 24 made of suitable synthetic or treated fabric or polymer sheet material, a junction ring 23 and a locking ring 22. The junction ring 23 provides a substantially rigid counterpart to features on inlet port 12 of heart pump 10 for establishing a mating connection therewith. Locking ring 22 binds the junction ring 23 and inlet port 12 together to provide a secure and non-thrombogenic blood flow passage from the interior of atrial cuff assembly 20 to pump 10. As further shown in phantom in FIG. 1 as well as in perspective in FIG. 2, an arched cross member 25 extends across a central opening 13 of the atrial cuff assembly 20 above the inlet port 12 to prevent tissue atrial tissue proximate to the inlet 12 from blocking the pump opening in the event that flow conditions (especially negative relative pressure conditions) draw atrial tissue 11 toward pump 10. Care should be taken to form cross member 25 of a material and texture that will reduce the risk of thombus formation when blood flows over the cross member.

Referring again to FIG. 1, a vascular tissue outlet connector 30 is adapted for fluid-tight connection to the outlet port 14 of pump 10. The illustrated vascular tissue outlet connector 30 is a vascular graft assembly that has a tubular flexible mesh or sheet body 34 formed as a vessel graft for suturing to the aorta or other vessel, which is joined to a substantially rigid junction ring 33 (FIG. 6) while a locking ring 32, corresponding to locking ring 22 of atrial cuff assembly 20, surrounds junction ring 33 and secures it to outlet port 14.

Each of the vascular tissue connectors 20, 30 may be provided in a range of sizes, i.e., having different size cuff 24 or graft body 34 fabric elements permanently attached to its respective junction ring 23, 33 (FIG. 6). The junction rings 22, 33 (FIG. 6), in turn, are of fixed and uniform diameter, or are, together with corresponding heart pumps, provided in a small number of sizes, matching those of the heart pump ports to which they attach. This allows the selection of different size graft connectors to be carried out during surgery while using a common size, or only a few sizes, of heart pump assembly.

In accordance with a principal aspect of the present invention, and using atrial cuff assembly 20 as an exemplary vascular tissue connector, the locking ring 22 employed for fastening vascular tissue connector 20 to inlet port 12 rotates freely with respect to the junction ring 23 and cuff 24. Furthermore, atrial cuff assembly 20 itself may rotate or change its orientation about port 12, and assume a fixed rotational orientation with respect to port 12 only when locking ring 22 has clamped the junction.

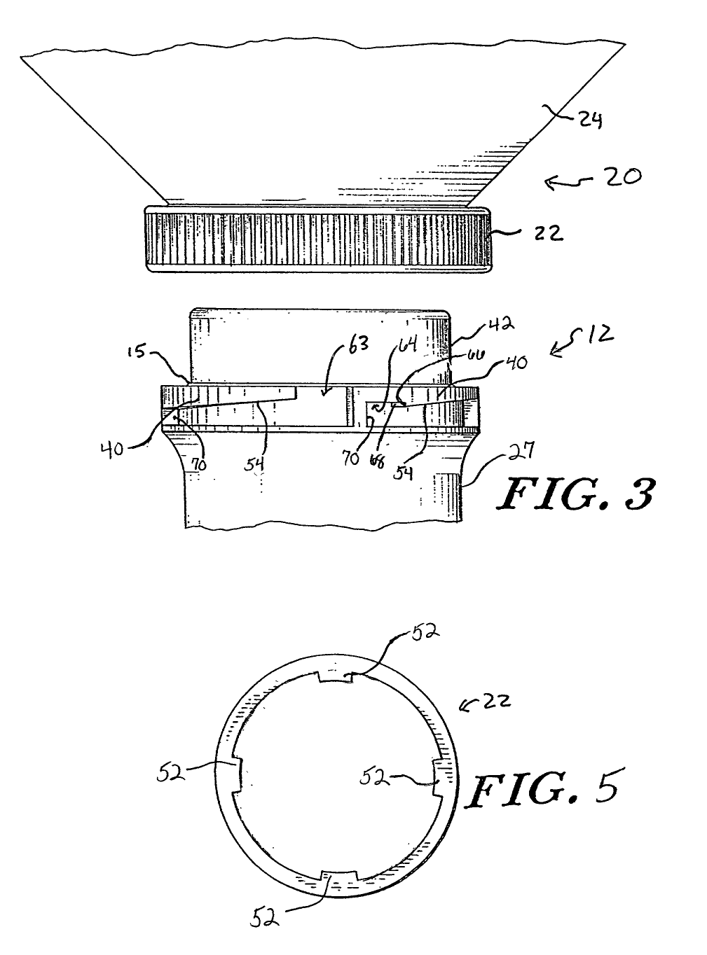

This operation will be better understood from FIGS. 3 and 4A-4C, which show a side view of port 12 and atrial cuff assembly 20 separated, and three successive partially cut away views of the assembly in various stages of coupling, respectively. As shown in FIG. 3, port 12 includes a flange portion 40 extending outward from, and contiguous with, an inlet region 27 of pump 10, and a neck extension portion 42 which runs as a liner or sleeve along the flow passage, extending from flange 40 and fitting to the interior of junction ring 23 of atrial cuff assembly 20 as shown in FIGS. 4A-4C. As shown on FIG. 4C, neck extension portion 42 can be configured to extend past junction ring 23 in the fully coupled position to provide a continuous blood flow surface. As further illustrated in FIGS. 4A-4C the blood contacting surfaces of port 12 are covered by a thin film 44 of a blood compatible polyurethane or suitable polymer coating, such as the hemocompatible material sold under the trade name ANGIOFLEX® by ABIOMED, Inc. of Danvers, Mass. Coating 44 preferably covers all active flow surfaces, and preferably also extends entirely over the inner end, and outer faces of neck extension portion 42. As further shown in FIGS. 4A-4C, an O-ring 15 can be fitted in the outer face or top surface of flange portion 40 and is compressed by junction ring 23.

Further details of atrial cuff assembly 20 can be described by reference to the cross-section of FIG. 4A. The atrial cuff assembly 20 includes a generally conical fabric skirt 24 which may, for example, have a multi-layer velour/film structure as shown in the aforesaid U.S. Pat. No. 5,084,064, and this skirt is attached to junction ring 23. The cuff or skirt 24 is preferably bonded with a filler bead 46 to junction ring 23, to eliminate the dead comer or recess from the otherwise sharp geometry of the junction so as to deter thrombus formation.

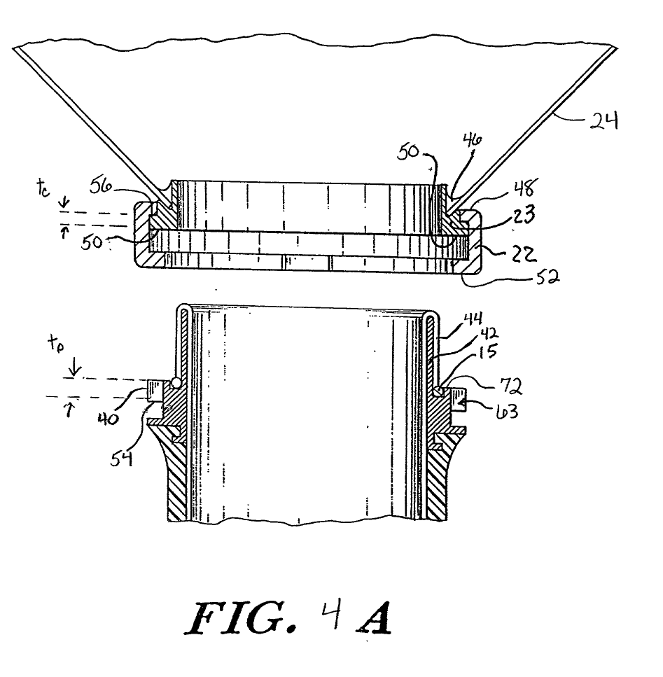

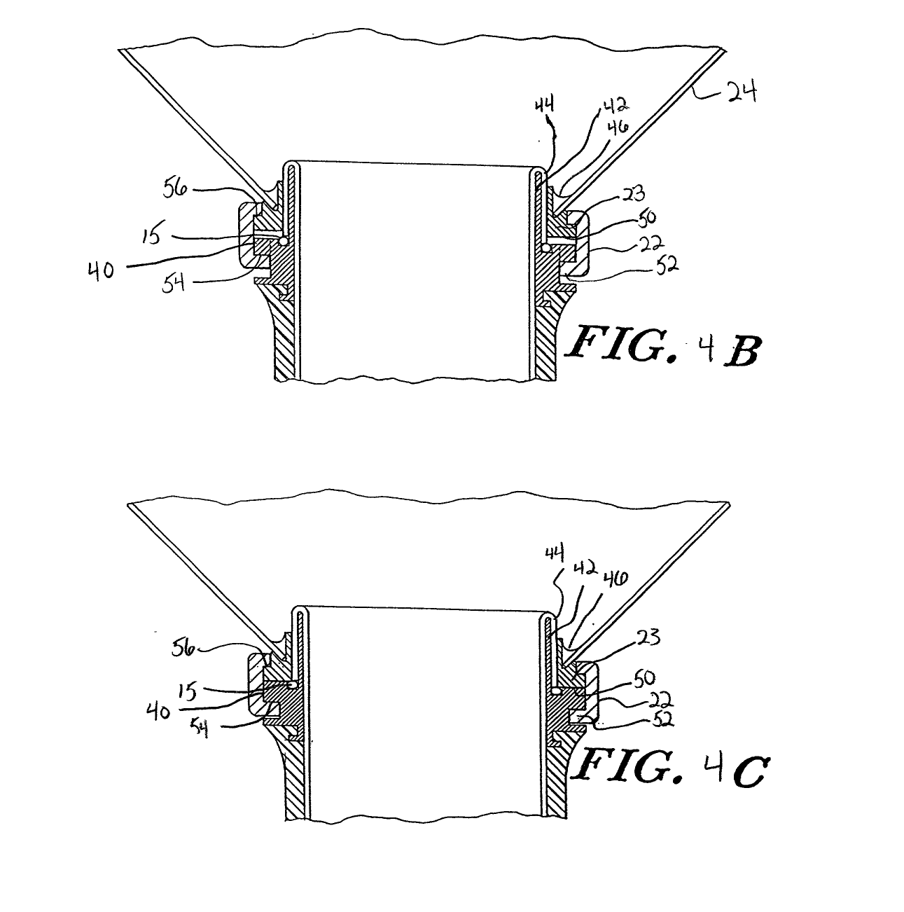

As further shown in the FIG. 4A, a top flange 48 of locking ring 22 interlocks with a flange 50 on junction ring 23 to permit locking ring 22 to lock down junction ring 23 while at the same time allowing locking ring 22 to freely rotate with respect to junction ring 23. As best seen in FIG. 5, locking ring 22 has a plurality of tabs 52 which protrude radially inward from the ring circumference. In the illustrated embodiment, there are four such tabs 52, each subtending about six or seven millimeters of arc around the circumference of locking ring 22 and protruding approximately five millimeters radially inward from the circumference. In the cross-section of FIGS. 4B-4C, tabs 52 are shown bearing against a lower surface 54 of flange 40 of outlet port 12, while the top flange 48 of locking ring 22 extends as a continuous annular surface over flange 50 on junction ring 23 and presses downward against an upper surface 56 of flange 50. Thus, in the regions where the tabs 52 appear, a cross section of locking ring 22 forms a C-shaped clamp ring connector.

In operation, locking ring 22 forms a C-shaped clamp that locks together atrial cuff assembly 20 and inlet port 12 by clamping together flange 50 on junction ring 23 of the atrial cuff assembly 20 and flange 40 of inlet port 12. Flange 40 on inlet port 12 has a thickness tₚ, and the flange 50 of the junction ring 23 on the atrial cuff assembly 20 has a thickness t_{c}, with the two flanges 40, 50 being squeezed together by rotation of the locking ring 22. The thickness t_{c} is constant, i.e., the flange 40 on atrial cuff assembly 20 is of constant thickness, while the flange 48 formed on inlet port 12 is either of increasing thickness tₚ, or can have a bottom surface 54 forming a helically configured ramp along the outer surface of inlet port 12. Thus, as the locking ring 22 is rotated, it draws the junction ring 23 of atrial cuff assembly 20 down tight against flange 40 on inlet port 12, seating it against an O-ring 15 which may be provided between the atrial cuff assembly 20 and inlet port 12.

A more detailed understanding of the variation in the thickness tₚ of flange 40 on inlet port 12 can be gained by viewing in sequence FIG. 3, which shows a side view of vascular tissue connector 20 (exemplified as an atrial cuff assembly) and inlet port 12, and FIGS. 4A-4C, which show cross-sections of connector 20 and port 12 (A) separated, (B) partially connected, and (C) fully connected. As shown in FIG. 3, inlet port 12 includes a plurality of radially protruding flanges 40. While FIG. 3 depicts two of four flanges 40 equally spaced about generally cylindrical inlet port 12, the number and location of the flanges 40 may be varied. Each flange 40 has a lower surface 54 that extends in a sloping or generally helical direction for at least a portion of its length, which in the illustrated embodiment is slightly less than a quarter of the circumferential perimeter of inlet port 12. A gap 63 can be provided between each of the successive flanges 40 to allow protrusions 52 on locking ring 22 to pass by flanges 40 and, by rotating the locking ring 22, engage bottom surface 54 of each flange 40.

The bottom surface 54 of each flange segment 40 slopes downwardly as a ramp to a notch or detent region 64, where a protrusion 52 passes the edge 66 of the ramp formed by bottom surface 54 of flange segment 40 and is prevented from moving backward by a detent face 68. An abutment or stop face 70 may also be provided further along the ramp to block the leading edge of the protrusion and prevent further forward rotation of locking ring 22. Alternatively, the ramp may simply continue to descend beyond the width of the locking ring to effectively jam and prevent over-rotation of the locking ring past its maximum pressure point.

The slope of the bottom face 54 of flange 40 is generally a shallow slope angle so that as a protrusion 52 slides along its surface it draws locking ring 22 downward with upper flange 48 of locking ring 22 forcing flange 50 of junction ring 23 against flange 40 on inlet port 12. This forcing action can compress O-ring 15 (if any). The relief provided by the cutout or detent 64 is a fraction of the total vertical run along the surface 54, so that as a protrusion 52 slides over edge 66, the compressive force on O-ring 15 is diminished slightly. This situation causes an elastic force to remain present by virtue of the partially compressed O-ring 15 to prevent the locking ring 22 from shifting axially along the connection, and consequently preventing a protrusion 22 from slipping over detent face 68 to unlock the connection and slide past edge 66. As an alternative to providing an elastic O-ring 15, the elastic force can also be provided by employing a material capable of some elastic deformation (though still rigid enough to substantially maintain its shape and the desired connection) in locking ring 22 and/or in flange 40. The elastic force provided by O-ring 15 can also cause protrusions 52 to “snap” or click into detent 64 to provide tactile and/or audio feedback to a surgeon operating the connector so that the surgeon knows that a positive locking configuration has been achieved.

The operation of the connecting ring 22 and its interaction with flanges 40 can further be seen by referring to FIGS. 4A to 4C in sequence. In FIG. 4A, atrial cuff assembly 20 is spaced apart from inlet port 12, and tabs 52 on locking ring 22 are aligned to slide through gaps 63 on inlet port 12. In FIG. 4B, atrial cuff assembly 20 and inlet port 12 are brought together, tabs 52 are passed through gaps 63, and locking ring 22 is turned to cause tabs 52 to engage surfaces 54 on flanges 40. In FIG. 4C, locking ring 22 is fully turned to cause tabs 52 to slide along surface 54 (which is shaped like a ramp in the illustrative embodiment) which in turn draws junction ring 23 into contact with flange 40 to create a sealing relationship between atrial cuff 20 and inlet port 12 at the junction ring-flange interface. As further illustrated in FIG. 4C, optional resilient O-ring 15 is compressed by contact with junction ring 23.

Advantageously, the atrial cuff assembly 20 may freely rotate with respect to the pump 10 and its inlet port 12 at all times prior to attaining the fully sealed configuration of FIG. 4C. In this way, after joining atrial cuff assembly 20 to a remaining portion of a patient's atrium, pump 10 is placed into position in the patient's chest cavity and the pump and atrial cuff assembly can be aligned relative to each other during positioning or repositioning of these components with respect to each other, to align them with respect to each other as illustrated in FIG. 4A. The sequence of FIGS. 4A to 4C can then be carried out, and locking ring 22 rotated to secure pump 12 to atrial cuff assembly 20 in a stress-free final aligned position without further rotating or disturbing the alignment of either the pump or the atrial cuff assembly.

Referring now to FIG. 6, a similar structure is provided for outlet port 14 and vascular tissue outlet connector 30 (illustrated as a vascular graft assembly) as was illustrated for inlet port 12 and atrial cuff assembly 20, and reference to specific structures on outlet port 14 or vascular graft assembly 30 does not indicate that corresponding structures illustrated for inlet port 12 or atrial cuff assembly 20 could not be used, and vice versa.

FIG. 6 shows a partial section in a plane containing the flow axis through the outlet port 14 and vascular graft assembly 30 of FIG. 1. The vascular graft assembly 30 includes a suturable sheet or fabric portion 34, which connects continuously to a rigid junction ring 33, which in turn is urged against outlet port 14 by a locking ring 32, which like inlet locking ring 22, can be formed of a biocompatible titanium alloy and can have a knurled or otherwise roughened exterior surface to allow the ring to be readily gripped and rotated.

In general, the top surface of flange 40, whether on inlet port 12 or outlet port 14, lies at a constant level forming a flat face which, for example, may seal against the junction ring 23 of the vascular tissue connector 20 as is shown in FIG. 4C. If the top of the flange is not to be a sealing or joining face, then preferably a separate bushing, seat or liner such as the polycarbonate insert ring 80 of FIG. 6 is provided to form a continuous blood flow junction surface 82. Insert ring 80 has an inner diameter precisely machined to fit against and form a continuous smooth surface with an inner flow face of junction ring 33. All of the blood contacting surfaces are preferably coated with polyurethane, ANGIOFLEX, or a similar medical polymer that can provide a smooth, non-toxic, non-thrombogenic blood contacting surface.

In addition, a top surface 84 can be provided on insert 80 that can be urged by pressure into a fluid-tight seal with junction ring 33. In that event, flange 40 or outlet port 14 (which can be formed integrally with pump 10) need not itself provide a sealing face, and may have a helically disposed flange 40 of constant thickness, with both its upper and lower faces lying along an incline similar to the thread of a lead screw. In the illustrated embodiment, an O-ring 17 is positioned between locking ring 32 and junction ring 33. A slight step or groove 86 in a face of junction ring 33 and a similar groove 88 in locking ring 32 serve to position O-ring 17, and the groove depth can be selected to form an appropriate compression gap. In this case, the O-ring 17 is situated between the locking ring 32 and the junction ring 33, and therefore performs no sealing function (in the illustrated embodiment, sealing is provided between the insert ring 80 and junction ring 33 at surface 84), but instead serves to distribute the contact pressure and avoid regions of high localized clamping stress as the two mating members are urged against each other by the rotation of the locking ring 32.

Because the illustrated locking rings function much like a V-ring clamp, the locking ring construction may be modified to accommodate other flat flanged elements, such as an outlet valve or a spacer ring for example, and to lock these elements in series between the pump and connector components. Still further, the connector of the invention can be characterized as two connector halves with one half being connected to or integral with a medical device and a second half being connectable to vascular tissue, and with the locking ring disposed on either half and being engageable with the other half. The invention being thus described and illustrated, further variations and modifications will occur to those skilled in the art and all such variations and modifications are considered to lie within the scope of the invention as defined by the claims appended hereto.

What is claimed is:

1. A connector for connecting a blood processing device to vascular tissue, the connector comprising:
a vascular tissue connecting element suturable to a portion of the cardiovascular system for providing a flow connection;
a junction ring affixed to the vascular tissue connecting portion to form a substantially shape retaining connecting element; and
a locking ring for locking the junction ring to a blood processing device, the locking ring including a coupling element configured to engage a port on the blood processing device so that rotation of the locking ring draws the junction ring to the port and locks the junction ring into a sealing relationship with the port;
wherein the locking ring is freely rotatable about the junction ring so that the locking ring can be rotated to lock the junction ring to the port without twisting the vascular tissue connecting element.

2. The connector of claim 1, wherein the coupling element comprises a plurality of protrusions directed radially inward for engaging a mating coupler of the port, the locking ring having a first surface positioned for bearing against the junction ring to exert an axial force thereon in a connecting direction and being rotatable to bring the protrusions into engagement with the port to cause the axial force.

3. The connector of claim 2, wherein the protrusions have a surface configured to engage the port to prevent further rotation of the locking ring when a desired axial force is exerted.

4. The connector of claim 2, wherein the protrusions are spaced at pairwise opposed positions about a circumference of the locking ring for engaging the port to exert a uniform axial force around the junction ring.

5. The connector of claim 2, further comprising a resilient sealing ring disposed between the junction ring and the first surface of the locking ring for compressibly distributing the axial force exerted on the junction ring.

6. The connector of claim 2, further comprising a resilient seal sealing ring disposed to form a fluid tight seal between the junction ring and the port as the axial force is applied.

7. The connector of claim 5, wherein the protrusions on the locking ring are configured to lock against rotation upon reaching an end rotation position and being urged axially in a direction away from connection, the resilient sealing element being disposed so as to urge the junction ring and locking ring in a direction away from connection when the junction ring is drawn to the port by rotation of the locking ring.

8. The connector of claim 6, wherein the protrusions on the locking ring are configured to lock against rotation upon reaching an end rotation position and being urged axially in a direction away from connection, the resilient sealing element being disposed so as to urge the junction ring and locking ring in a direction away from connection when the junction ring is drawn to the port by rotation of the locking ring.

9. The connector of claim 1, wherein the vascular tissue connecting element is an atrial cuff.

10. The connector of claim 9, wherein the junction ring and locking ring are configured to connect to an inlet port of a blood pump.

11. The connector of claim 9, further comprising a cross member extending across a central opening of the junction ring, the cross member configured to prevent vascular tissue from collapsing into the central opening.

12. The connector of claim 11, wherein the cross member is affixed to the junction ring and comprises an arched cross member extending in an upstream direction across the central opening.

13. The connector of claim 1, wherein the vascular tissue connecting element is a vascular graft element configured to connect to an artery.

14. The connector of claim 13, wherein the junction ring and locking ring are configured to connect to an outlet port of a blood pump.

15. A method for connecting a blood pumping device to a cardiovascular system, the method comprising:
joining a prosthetic tissue ending having a junction ring to a portion of the cardiovascular system to provide a flow connection, the junction ring forming a substantially rigid termination of defined size and shape;
aligning the junction ring with a port on a blood pumping device;
rotating a locking ring to sealingly connect the port on the blood pumping device to the junction ring, the locking ring permitting free relative rotation between the port and the junction ring until the sealing connection is achieved.

16. The method of claim 15, wherein the method further comprises disposing a sealing ring between the junction ring and the port to seal the connection between the junction ring and the port.

17. The method of claim 15, wherein the step of aligning includes rotating a first one of the junction ring and the port with respect to a second one of the junction ring and the port.

18. The method of claim 15, wherein the step of rotating the locking ring further includes aligning protrusions provided on the locking ring to engage a coupling element.

19. The method of claim 15 wherein the method further comprises joining the prosthetic tissue ending to a patient's heart.

20. The method of claim 15 wherein the method further comprises joining the prosthetic tissue ending to a patient's blood vessels.

21. A medical quick connector for making a fluid connection between vascular tissue and a medical device, the connector comprising:
a first connector half having a mating end and a connector engaging end including a junction ring affixed thereto and a locking ring rotatably coupled to the junction ring;
a second connector half having mating end and a connector engaging end having a locking ring coupling element;
wherein a first one of the mating ends of the first and second connector halves is adapted to mate with the vascular tissue and a second one of the mating ends of the first and second connector halves is adapted to mate with the medical device; and
whereby the first connector half can be aligned with the second connector half, placed into engagement with the second connector half, then locked to the second connector half by rotation of the locking ring without rotation of either the first or second connector halves.

22. The connector of claim 21, wherein a first one of locking ring and the locking ring coupling element includes one or more protruding elements and a second one of locking ring and the locking ring coupling element includes one or more protrusion receiving elements configured to lock the first and second connector halves together in a fluid tight seal upon rotation of the locking ring with respect to the locking ring coupling element.

23. The connector of claim 22, wherein the one or more protrusion receiving elements include a detent for locking the one or more protrusions to resist rotation of the locking ring with respect to locking ring coupling element when a fluid tight seal has been achieved.

24. The connector of claim 23, wherein a bias element urges the one or more protrusions to engage the detent.

25. The connector of claim 24, wherein the bias element is an elastic O-ring disposed between the connector halves.

26. The connector of claim 23, wherein the urging of the one or more protrusions to engage the detent provides tactile feedback to a connector operator that a fluid tight locking seal has been made.

27. The connector of claim 23, wherein the urging of the one or more protrusions to engage the detent provides audio feedback to a connector operator that a fluid tight locking seal has been made.

28. The connector of claim 21, wherein the first one of the mating ends of the first and second connector halves includes a vascular graft.

29. The connector of claim 21, wherein the first one of the mating ends of the first and second connector halves includes an atrial cuff.

30. The connector of claim 21, wherein the second one of the mating ends of the first and second connector halves is integral with a medical device.

31. A connector for connecting a blood pumping device to vascular tissue, the connector comprising:
a vascular tissue connecting element suturable to a portion of the cardiovascular system for providing a flow connection;
a junction ring defining a central opening and affixed to the vascular tissue connecting portion to form a substantially shape retaining connecting element;
a cross member extending across a central opening of the junction ring, the cross member configured to prevent vascular tissue from collapsing into the central opening and
a locking ring for locking the junction ring to a blood pumping device.

32. The connector of claim 31, wherein the locking ring includes a coupling element configured to engage a port on the blood pumping device so that rotation of the locking ring draws the junction ring to the port and locks the junction ring into a sealing relationship with the port.

33. The connector of claim 32, wherein the locking ring is freely rotatable about the junction ring so that the locking ring can be rotated to lock the junction ring to the port without twisting the vascular tissue connecting element.

34. The connector of claim 31, wherein the coupling element comprises a plurality of protrusions directed radially inward for engaging a mating coupler of the port, the locking ring having a first surface positioned for bearing against the junction ring to exert an axial force thereon in a connecting direction and being rotatable to bring the protrusions into engagement with the port to cause the axial force.

35. The connector of claim 34, wherein the protrusions have a surface configured to engage the port to prevent further rotation of the locking ring when a desired axial force is exerted.

36. The connector of claim 34, wherein the protrusions are spaced at pairwise opposed positions about a circumference of the locking ring for engaging the port to exert a uniform axial force around the junction ring.

37. The connector of claim 34, further comprising a resilient sealing ring disposed between the junction ring and the first surface of the locking ring for compressibly distributing the axial force exerted on the junction ring.

38. The connector of claim 34, further comprising a resilient seal sealing ring disposed to form a fluid tight seal between the junction ring and the port as the axial force is applied.

39. The connector of claim 37, wherein the protrusions on the locking ring are configured to lock against rotation upon reaching an end rotation position and being urged axially in a direction away from connection, the resilient sealing element being disposed so as to urge the junction ring and locking ring in a direction away from connection when the junction ring is drawn to the port by rotation of the locking ring.

40. The connector of claim 38, wherein the protrusions on the locking ring are configured to lock against rotation upon reaching an end rotation position and being urged axially in a direction away from connection, the resilient sealing element being disposed so as to urge the junction ring and locking ring in a direction away from connection when the junction ring is drawn to the port by rotation of the locking ring.

41. The connector of claim 31, wherein the vascular tissue connecting element is an atrial cuff.

42. The connector of claim 41, wherein the junction ring and locking ring are configured to connect to an inlet port of the blood pumping device.

43. The connector of claim 31, wherein the cross member is affixed to the junction ring and comprises an arched cross member extending in an upstream direction across the central opening.

44. A medical device and connector system for attaching the medical device to a cardiovascular system, comprising:
a port formed on the medical device;
a connector element having a vascular tissue connecting portion and a junction ring attached to the vascular tissue connecting portion and having a predefined shape defining a central opening;
a locking ring provided on a first one of the port and the connector element;
a locking ring engaging element provided on a second one of the port and the connector element;
wherein the locking ring is freely rotatable about the junction ring so that the locking ring can be rotated to lock the junction ring to the port without twisting the connecting element.

45. The connector of claim 44, wherein the locking ring includes a plurality of protrusions for engaging the locking ring engaging element.

46. The connector of claim 45, wherein the protrusions are spaced at pairwise opposed positions about a circumference of the locking ring for engaging the locking ring engaging element to exert a uniform axial force around the port and the junction ring and draw them together into a sealing relationship upon rotation of the locking ring.

47. The connector of claim 46, further comprising a resilient seal sealing ring disposed in a series pressure relationship between the junction ring and the port for compressibly distributing the axial force applied.

48. The connector of claim 47, wherein the protrusions on the locking ring are configured to lock against rotation upon reaching an end rotation position and being urged axially in a direction away from connection, the resilient sealing element being disposed so as to urge the junction ring and locking ring in a direction away from connection when the junction ring is drawn to the port by rotation of the locking ring.

49. The connector of claim 44, wherein the port includes an extending portion configured to extend through the central opening of the connector element to provide a continuous blood flow surface therethrough.

50. The connector of claim 49, wherein the vascular tissue connecting portion is an atrial cuff.

51. The connector of claim 50, wherein the port is an inlet port.

52. The connector of claim 50, wherein the junction ring includes a cross member extending across a central opening of the junction ring, the cross member configured to prevent vascular tissue from collapsing into the central opening.

53. The connector of claim 44, wherein the port includes an insert ring shaped to provide a continuous blood flow surface from the port to the junction ring.

54. The connector of claim 53, wherein the vascular tissue connecting portion is a vascular graft element configured to connect to an artery.

55. The connector of claim 54, wherein the port is an outlet port.
